(19) United States
(12) Patent Application Publication
(10) Pub. No.: US 2007/0196324 A1
(43) Pub. Date: Aug. 23, 2007

(54) Cellulite skin treatment methods and compositions

(76) Inventors: Candace R. Keefe, Laguna Beach, CA (US); Michelle Arth, Aliso Viejo, CA (US); Antoaneta Abbruzzese, Aliso Viejo, CA (US); Pierre Bottiglieri, Conthey-Sion (CH)

Correspondence Address: WAGNER, ANDERSON & BRIGHT, LLP, 3541 OCEAN VIEW BLVD, GLENDALE, CA, 91208, US

(21) Appl. No.: 11/701,030

(22) Filed: Jan. 31, 2007

Related U.S. Application Data
(60) Provisional application No. 60/764,740, filed on Feb. 1, 2006.

(51) Int. Cl. A61K 31/74

(52) U.S. Cl. 424/78.03

(57) ABSTRACT

Methods for treating cellulite-affected areas on a human body include applying, to affected skin areas, while dry, a skin scrub that includes certain oils, menthone glycerin acetal, and certain organic extracts, then removing the scrub, moistening the affected areas, and applying a skin cream that includes caffeine, certain organic extracts and oils, and menthone glycerin acetal.

This application claims the benefit of provisional application Ser. No. 60/764,740, filed Feb. 1, 2006.

### FIELD OF THE INVENTION

Cellulite comprises abnormal subcutaneous deposits of irregular fatty masses under the skin. The fatty masses result in irregular disturbances in the skin's normally smooth contours. Methods for treating cellulite involve topical application of a variety of chemical compositions, massages, exercise and dietary actions. Mechanical devices and body wraps have also been used without significant success, and usually provide only temporary improvement. This invention provides compositions and methods for topical application of these compositions to affected skin areas.

### SUMMARY OF THE INVENTION

The present invention overcomes the shortcomings of the prior art by providing a daily synergistic 2-step system that visually diminishes the effects of cellulite dimpling. Step 1 comprises applying a cellulite scrub to dry skin, prior to showering, in the cellulite-affected areas (thighs, hips, buttocks, arms), preferably in circular motions to increase microcirculation. Step 2 comprises applying a contouring cellulite cream liberally to the affected areas until absorbed.

More particularly, the invention comprises the topical application of a skin scrub formulation onto the cellulite-afflicted areas of the skin. The scrub is massaged into dry skin for an effective time period to stimulate microcirculation. Subsequently, the scrub is flushed from the skin with water, leaving the skin prepared for the contouring cream. Thereafter, a contouring cream is massaged over the affected areas in circular motions, which also may be applied for an effective time to further enhance the benefits of the pre-shower scrub.

The treatment is preferably carried out at least once per day. After six weeks of treatment, significant improvement in the cellulite appearance, tactile roughness and skin dryness of the affected area(s) occurs. Other improvements may include decreased appearance of cellulite, firmer, toned, moisturized, smoother, less dimpled skin, increased skin elasticity and suppleness, reduced ‘orange peel’ look, area appears less heavy. These results continue to improve at 8 weeks and 12 weeks. Contouring and slimming of the affected skin area(s) occurs at 8 and 12 weeks.

The scrub composition comprises active ingredients of 15 wt % to 40 wt % moisturizing oils, 1.000 wt % to 5 wt % menthone glycerin acetal, 0.001 wt % to 1.000 wt % horse chestnut extract, 0.001 to 1.000 wt % guarana extract, and inactive ingredients in an amount to make up a 100% composition.

The skin cream composition comprises 4.0200 wt % to 5.000 wt % caffeine, 3.0000 wt % to 3.1000 wt % blupleurum falcatum extract, 0.001 wt % to 3.000 wt % Coenzyme A, 0.001 wt % to 1.000 wt % horse chestnut extract, 3.000 wt % to 5.000% menthone glycerin acetyl, 0.1000 wt % to 1.000 wt % carnitine, and inactive ingredients in an amount to make up a 100% composition.

The following is a quantitative formula for the skin scrub of the invention:

| Ingredients | Percentage |
|---|---|
| Sorbitan Stearate | 17.500 |
| Helianthus Annuus (Sunflower) seed Oil | 15.000 |
| Sucrose | 15.000 |
| Glycine Soja (Soybean) Oil | 10.992 |
| Hydrated Silica | 10.000 |
| Magnesium Sulfate | less than 8.000 |
| Silica | less than 8.000 |
| Dimethicone | less than 8.000 |
| PEG-100 Stearate | 5.500 |
| Olea Europaea (Olive) Fruit Oil | 5.000 |
| Simmondsia Chinensis (Jojoba) seed Oil | 5.000 |
| Butyrospermum Parkii (Shea Butter) | 2.000 |
| Sea Salt | 2.000 |
| Citrus Grandis (Grapefruit) Peel Oil | 1.000 |
| Menthone Glycerin Acetal | 1.000 |
| Tocopheryl Acetate (Vitamin E Acetate) | 1.000 |
| Citrus Aurantium Dulcis (Orange) Oil | 0.500 |
| Propylparaben | 0.500 |
| Carthamus Tinctorius (Safflower) Seed Oil | 0.001 |
| Theobroma Cacao (Cocoa) Extract | less than .001 |
| Rosemarinus Officinalis (Rosemary) Leaf Extract | 0.001 |
| Camellia Oleifera Leaf Extract | 0.001 |
| Arnica Montana Leaf Extract | 0.001 |
| Ruscus Aculeatus (Butchers Broom) Root Extract | 0.001 |
| Centella Asiatica Extract | 0.001 |
| Paullinia Cupana (Guarana) Seed Extract | 0.001 |
| Aesculus Hippocastanum (Horse Chestnut) Extract | 0.001 |

A quantitative formulation useful as a skin cream of the invention is as follows:

| Ingredients | Percentage |
|---|---|
| Water | 68.3130 |
| Menthone Glycerin Acetal | 5.0000 |
| Caffeine | less than 7.5000 |
| PEG-12 Glyceryl Distearate | less than 3.0000 |
| Blupleurum Chinensis (Blupleurum Falcatum) Extract | 3.0000 |
| Coenzyme A | less than 3.0000 |
| Caprylic/Capric Triglyceride | 3.0000 |
| Silicone Gel | 2.0000 |
| Cyclomethicone | 2.0000 |
| Glyceryl Stearate | 2.0000 |
| PEG-1000 Stearate | 2.0000 |
| Glyceryl Stearate | |
| Sodium Benzoate | 2.0000 |
| Cetyl Alcohol | 1.5000 |
| Stearyl Alcohol | 1.5000 |
| Dimethicone | 1.0000 |
| Phenoxyethanol | 1.0000 |
| Triethanolamine | 0.6020 |
| Acrylates/c10-30 Alkyl Acrylate Crosspolymer | 0.4000 |
| Methylparaben | 0.2500 |
| Butylene Glycol | 0.1550 |
| Carnitine | 0.1000 |
| Propylparaben | 0.1000 |
| Lecithin | 0.0300 |
| Hydrolyzed Wheat Protein | 0.0200 |
| Carbomer | 0.0050 |
| Esculin | 0.0020 |
| Salicylic Acid | 0.0020 |
| Centella Asiatica (Gotu Kola) Extract | 0.0001 |
| Aesculus Hippocastanum (Horse Chestnut) Extract | 0.0001 |
| Lanacryst | 0.0001 |
| Aloe Barbadensis (Aloe) Leaf Extract | 0.0001 |
| Arnica Montana Flower Extract | 0.0001 |
| Calendula Officinalis Flower Extract | 0.0001 |
| Camellia Olefera (Green Tea) Leaf Extract | 0.0001 |
| Carthamus Tinctorius (Safflower) Seed Oil | 0.0001 |
| Helianthus Annuus (Sunflower) Seed Oil | 0.0001 |
| Olea Europaea (Olive) Fruit Oil | 0.0001 |

The skin scrub may be made in multiple phases. The first phase comprises mixing, in a dry tank, soybean oil, sunflower oil, olive oil, jojoba oil, grapefruit oil and orange oil at room temperature.

In Phase 2, slowly add hydrated silica with continuous mixing, until the silica is entirely dissolved into the phase 1 oils. Heat to 75° C.

For phase 3, in a separate vessel, mix together sorbitan stearate, PEG-100 stearate, shea butter and propylparaben. Heat the mixture to 75° C. until the ingredients are melted, then slowly add to the phase 1/phase 2 mixture. Cool the materials to 50° C.

Phase 4 comprises adding sucrose and sea salt and Vitamin E Acetate, while maintaining a temperature of 50° C.

Phase 5 comprises mixing into the existing batch magnesium sulfate, silica and dimethicone at 50°. Once the material dissolves and the batch has a smooth consistency, cool to 45° C.

In phase 6, add cocoa extract, guarana extract, gotu kola extract, horse chestnut extract, arnica extract, butcher's broom extract, green tea extract and rosemary leaf extract. Each of the ingredients is added one by one until each ingredient is mixed well into the main batch.

In phase 7, add menthone glycerin acetal at 45° C. Mix well until the overall batch is homogeneous. Subsequently, the overall batch may be poured while warm into containers. No water or moisture of any kind should come into contact with the batch.

The skin cream formulation may be made in the following phases:

In phase 1, to a main vessel, add a prescribed amount of water, then add acrylates/c10-30 alkyl acrylate crosspolymer and carbomer. Mix until the mixture is uniform.

Phase 2: slowly add caffeine, PEG-12 glyceryl distearate.

Phase 3: to the main vessel, add sodium benzoate, methylparaben and aloe vera leaf extract. The ingredients should be added while heating to 70° C., and the temperature is maintained until all solids are dissolved.

Phase 4 begins in a separate vessel. Add the following ingredients by continuous mixing and heat to 70° C.: lecithin, butylene glycol caprylic/capric triglyceride, dimethicone, cyclomethicone, cetyl alcohol, stearyl alcohol, glyceryl stearate, PEG-100 stearate, safflower oil, sunflower oil, olive oil, propylparaben, salicylic acid and silicone gel.

Upon dissolution of the solids, the phase 4 batch may be added to the main batch at 70° C. with constant mixing. The mixing should continue while the mixed batches are cooled to 40° C. When 40° C. is reached, phase 5 comprises adding triethanolamine, carnitine, phenoxyethanol, bluplerum falcatum, caffeine, coenzyme A, gotu kola extract, calendula extract, arnica extract, lanacryst, horse chestnut, green tea extract, menthone glycerin acetal, hydrolyzed wheat protein and esculin.

The above ingredients should be added one by one, until each one has been uniformly mixed into the overall batch. The temperature may be cooled to 35° C., and the product may be poured into containers.

Although the above products may be physically characterized as a cream, lotion, gel, tonic, balm and the like, references to the skin cream of the invention are deemed to encompass the physical form of all of the above without being limited to any particular form. The above materials, particularly when used in the processing steps described above, will reduce the effect of cellulite on the skin and create a more pleasing appearance.

Following the synergistic two-step method of this invention helps to visually diminish the effects of cellulite. The first step uses a botanically-based skin scrub that is applied before showering to visually tone, tighten and prepare skin for step 2. The scrub is applied to affected areas, most commonly the thighs, hips, buttocks and arms, in circular motions. Subsequently, the scrub is rinsed away under a warm water shower or bath.

After showering, the contouring cellulite cream is applied liberally, preferably in circular motions, to the affected areas until absorbed into the skin. After 6-8 weeks of daily use following the above regimen, the cellulite affected areas develop smoothly contoured skin without significant adverse redness or irritation of the skin. These results tend to improve after consistent practice of the invention for 8-12 weeks.

We claim:

1. A topical skin scrub composition for treating the appearance of cellulite having the active ingredients comprising: 15 wt % to 40 wt % moisturizing oils, 1.000 wt % to 5 wt % menthone glycerin acetal, 0.001 wt % to 1.000 wt % horse chestnut extract, 0.001 wt % to 1.000 wt % guarana extract, and inactive ingredients in an amount to make up a 100% composition.

2. A topical skin cream composition for treating the appearance of cellulite having the active ingredients comprising: 4.0200 wt % to 5.000 wt % caffeine, 3.0000 wt % to 3.1000 wt % blupleurum falcatum extract, 0.001 wt % to 3.000 wt % Coenzyme A, 0.001 wt % to 1.0000 wt % horse chestnut extract, 3.000 wt % to 5.000 wt % menthone glycerin acetyl, 0.1000 wt % to 1.000 wt % carnitine, and inactive ingredients in an amount to make up a 100% composition.

3. A method for diminishing the appearance of cellulite comprising:
a. applying an effective amount of a skin scrub composition to a dry selected area of the skin affected with the appearance of cellulite, said skin scrub composition comprising a combination of 15 wt % to 40 wt % moisturizing oils, 1.000 wt % to 5 wt % menthone glycerin acetal, 0.001 wt % to 1.000 wt % horse chestnut extract, 0.001 wt % to 1.000 wt % guarana extract, and inactive ingredients in an amount to make up a 100% composition;
b. removing said skin scrub from said selected area; and,
c. moistening said selected area and applying a skin cream to the moistened area, said skin cream comprising 4.0200 wt % to 5.000 wt % caffeine, 3.0000 wt % to 3.1000 wt % blupleurum falcatum extract, 0.001 wt % to 3.000 wt % Coenzyme A, 0.001 wt % to 1.0000 wt % horse chestnut extract, 3.000 wt % to 5.000 wt % menthone glycerin acetyl, 0.1000 wt % to 1.000 wt % carnitine, and inactive ingredients in an amount to make up a 100% composition.

4. The method of claim 3 wherein after step b, the selected area is cleansed.
